(19) United States
(12) Patent Application Publication
(10) Pub. No.: US 2007/0243250 A1
(43) Pub. Date: Oct. 18, 2007

(54) Oral Dosage Formulations and Methods of Preparing the Same

(75) Inventor: Grant Wayne Heinicke, Rockaway, NJ (US)

Correspondence Address: CANTOR COLBURN, LLP, 55 GRIFFIN ROAD SOUTH, BLOOMFIELD, CT, 06002, US

(73) Assignee: Actavis Group PTC hf, Hafnarfjordur (IS)

(21) Appl. No.: 11/736,113

(22) Filed: Apr. 17, 2007

Related U.S. Application Data
(60) Provisional application No. 60/792,442, filed on Apr. 17, 2006.

(51) Int. Cl. A61K 9/24

(52) U.S. Cl. 424/465

(57) ABSTRACT

A method of optimizing a dissolution profile for a selected active agent dosage form, comprises combining a first amount of a first ensemble of pulsed-release pellets having a first dissolution profile with a first T50 and a second amount of a second ensemble of pulsed-release pellets having a second dissolution profile with a second T50 to produce a combination ensemble of pellets having a combination dissolution profile of a combination slope, wherein the combination slope corresponds to a single phase release, and wherein the combination slope is greater than 10% lower than the slope of the first dissolution profile and greater than 10% lower than the slope of the second dissolution profile, and wherein the first ensemble of pellets and the second ensemble of pellets comprise a core having disposed thereon a core composition layer, the core composition layer comprising the active agent, and a pulsed-release coating disposed on the core composition layer.

### CROSS-REFERENCE TO RELATED APPLICATIONS

This application claims the benefits of U.S. Provisional Patent Application Ser. No. 60/792,442 filed Apr. 17, 2006, which is fully incorporated herein by reference.

### BACKGROUND

Sustained-release dosage forms for oral administration, designed to deliver a pharmacologically active agent over an extended time period, are well known. Advantages of sustained-release formulations include longer, more consistent in vivo blood levels of active pharmaceutical ingredient, lower initial bursts of active pharmaceutical ingredient, and increased therapeutic benefits by eliminating fluctuations in serum active pharmaceutical ingredient levels. The advantages also include increased patient compliance and acceptance by reducing the required number of administrations.

Diltiazem hydrochloride, used principally for its calcium channel blocking properties, finds application in the treatment of angina pectoris and hypertension. Because diltiazem has a short half-life in the bloodstream, frequent dosing or sustained-release dosage forms are often preferred.

U.S. Pat. No. 5,834,024 describes diltiazem pellet formulations having controlled-release over a 24 hour period. The formulations comprise a population of short lag pellets and a population of long lag pellets that, when combined, give a biphasic release profile in vitro. By combining short lag and long lag pellets into a single formulation, the release of diltiazem is controlled over a twenty four hour period.

While the foregoing diltiazem formulations are well-suited for their intended purpose, there nevertheless remains a need for dosage forms of diltiazem and other active agents having desired release profiles.

### SUMMARY

A method of optimizing a dissolution profile for a selected active agent dosage form comprises combining a first amount of a first ensemble of pulsed-release pellets having a first dissolution profile with a first T50 and a second amount of a second ensemble of pulsed-release pellets having a second dissolution profile with a second T50 to produce a combination ensemble of pellets having a combination dissolution profile of a combination slope, wherein the combination slope corresponds to a single phase release, and wherein the combination slope is greater than 10% lower than the slope of the first dissolution profile and greater than 10% lower than the slope of the second dissolution profile, wherein the first ensemble of pellets and the second ensemble of pellets comprise a core having disposed thereon a core composition layer, the core composition layer comprising the active agent, and a pulsed-release coating disposed on the core composition layer.

A method of increasing patient compliance comprises providing an active agent dosage form to a human patient in need thereof, wherein the dosage form is produced by the above-described method.

An active agent dosage form comprises a first amount of a first ensemble of pulsed-release pellets comprising a first average pulsed-release coating weight and having a first dissolution profile with a first T50, and a second amount of a second ensemble of pulsed-release pellets comprising a second average pulsed-release coating weight and having a second dissolution profile with a second T50, wherein the first ensemble and the second ensemble are combined to form a combination ensemble of pellets having a combination dissolution profile of a combination slope, wherein the first average pulsed-release coating weight and the second average pulsed-release coating weight differ by 1 wt % or greater based on the weight of the coating material, wherein the combination slope corresponds to a single phase release, and wherein the combination slope is greater than 10% lower than the slope of the first dissolution profile and greater than 10% lower than the slope of the second dissolution profile, and wherein the first ensemble of pellets and the second ensemble of pellets comprise a core having disposed thereon a core composition layer, the core composition layer comprising the active agent, and the pulsed-release coating disposed on the core composition layer.

The above-described and other features will be appreciated and understood by those skilled in the art from the following detailed description and appended claims.

### BRIEF DESCRIPTION OF THE DRAWINGS

FIG. 1 shows an idealized pulsed-release profile.

FIG. 2 shows the release of individual pellets taken from an ensemble of pellets at a given coat weight.

FIG. 3 shows release from a 40 mg equivalent and 300 mg equivalent of pellets in a Dissolution Apparatus II.

FIG. 4 shows the sum of the releases from individual pellets from FIG. 1.

FIG. 5 shows the release from a 7 wt % coating weight ensemble, a 15 wt % coating weight ensemble, and a combination ensemble formed by mixing the 7 wt % and 15 wt % ensembles in a 1:1 blend.

FIG. 6 shows the release profiles for pellets having four different coating weights and theoretical release profiles for four blends each comprising two different coating weights of pellets.

FIG. 7 shows the release profiles for pellets having three different coating weights and theoretical release profiles for two blends each comprising two different coating weights of pellets.

FIG. 8 shows the release profiles for pellets having three different coating weights and theoretical release profiles for two blends each comprising two different coating weights of pellets.

### DETAILED DESCRIPTION

An active agent is a species that, when administered to a patient, confers, directly or indirectly, a physiological effect on the patient. An indirect physiological effect, for example, includes the effect of a metabolite of the active agent. Active agent includes solvates (including hydrates) of the free compound or salt, crystalline and non-crystalline forms, as well as various polymorphs. For example, an active agent can include all optical isomers and all pharmaceutically acceptable salts thereof either alone or in combination. “Pharmaceutically acceptable salts” includes derivatives of the active agent, wherein the active agent is modified by making non-toxic acid or base addition salts thereof, and further refers to pharmaceutically acceptable solvates, including hydrates, of such compounds and such salts.

By “oral dosage form” is meant to include a unit dosage form prescribed or intended for oral administration. An oral dosage form comprises a plurality of subunits such as, for example, pellets, packaged for administration in a single dose such as a capsule, sachet or a tablet. The oral dosage form optionally comprises a loading dose of the active agent in the form of, for example, a coating or a matrix. A loading dose is an immediate release portion of the dosage form.

By “releasable form” is meant to include immediate-release, controlled-release, and sustained-release forms. Certain release forms can be characterized by their dissolution profile. By “immediate-release”, it is meant a conventional or non-modified release in which greater than or equal to about 70% of the active agent is released within 1 hour, specifically within 30 minutes of the initiation of dissolution. By “controlled-release” it is meant a dosage form in which the release of the active agent is controlled or modified over a period of time. “Sustained-release” or “extended-release” include the release of the active agent for an extended period of time so that the dosage frequency can be reduced.

Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

Dissolution of single pellets is performed by a suitable method, such as, for example, circulating 0.1N HCl dissolution media through a sample cell containing a single pellet in line with a HPLC detector. Drug release is monitored by a chart recorder. Response of the chart recorder may be calibrated with standard solutions of drug. The volume of the loop is between 5 and 50 mL.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. By the term “highly acidic pH” is meant a pH of about 1 to about 4. By the term “less acidic to basic pH” is meant a pH of greater than about 4 to about 7.5, preferably about 6 to about 7.5. A pH of about 1.2 can be used to simulate the pH of the stomach. A pH of about 6 to about 7.5, specifically about 6.8, can be used to simulate the pH of the intestine.

Release forms may also be characterized by their pharmacokinetic parameters. “Pharmacokinetic parameters” are parameters that describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

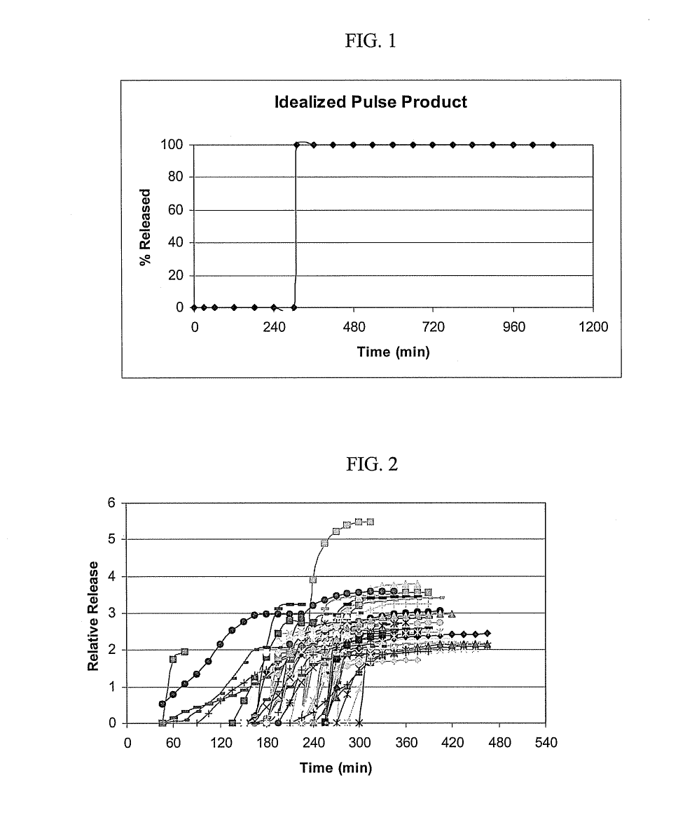

As used herein, an ensemble of “pulsed-release” pellets comprises a plurality of pellets, wherein at least about 80% of the individual pulsed-release pellets in the ensemble exhibits a lag time of substantially no release of active agent followed by immediate release of the active agent. As used herein, an ensemble of pulsed-release pellets is a population of pulsed-release pellets having substantially the same composition, e.g., a population of pellets coated so as to have the same coating weight. As shown in FIG. 1, an ideal pulsed-release active agent delivery ensemble should release the active pharmaceutical ingredient completely and rapidly after a lag time. The ensemble also has a lag time of essentially no release followed by release of the active agent, where said release results in a dissolution profile of a given slope. However, because the ensemble is comprised of a distribution of individual pellets, release after the lag time may occur over a period of time that is somewhat longer than is typical for immediate release, or for the individual pellets themselves. However, the release of the ensemble may not be the desired extended, sustained, or controlled-release due to the pulsed-release nature of the formulation. The T50 of an ensemble of pellets is the time for the ensemble to release 50% of the active pharmaceutical ingredient, measured as the halfway point between the time axis and the extent of release. In contrast to an idealized pulsed-released profile which should be square at the top of the curve, the measured pulsed-release profiles exhibit a rounding-off at the top of the dissolution curve.

Measurement of active agent release from single pellets has yielded the dissolution profiles shown in FIG. 2. As shown in FIG. 2, once release starts, most (e.g., greater than 80%) of the pellets have a very rapid release. Without being held to theory, it is believed that the rapid release is caused by bursting of the coating to relieve pressure build up as water soaks into the center of the pellet. At some pressure, the coat bursts and the contents are released substantially immediately. What FIG. 2 illustrates is that the variability in time to release between single pulsed-release pellets can be significant, however, most pellets are immediate release after a lag time. For example, in a single ensemble of pellets having a coating weight of 11 wt %, release times of 2.5 to 5 hours were determined for the majority of the individual pellets within the ensemble.

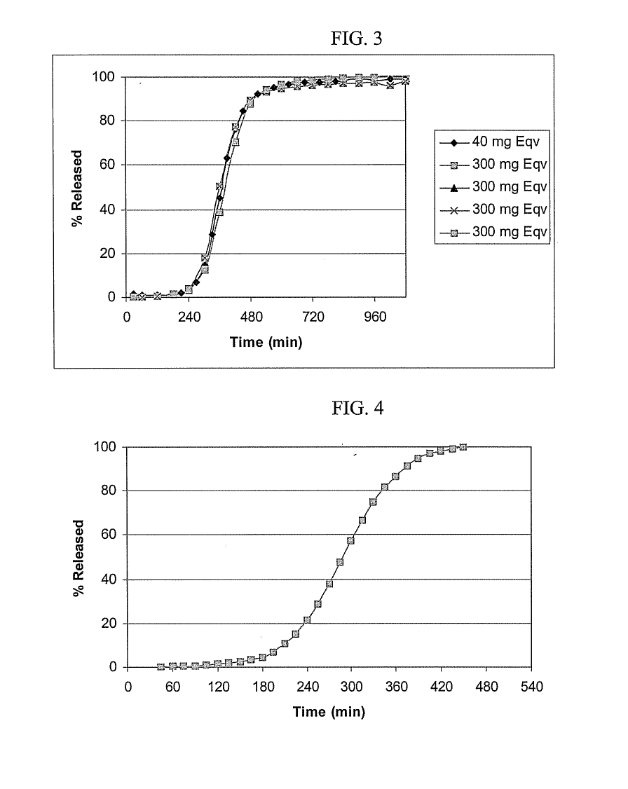

FIG. 4 shows that the sum of the individual plots from FIG. 2 gives a release profile that is very similar to that determined for the entire ensemble of pellets as a whole as shown in FIG. 3. Therefore, the profile is the sum of the distribution of lag times of the individual pellets and the overall ensemble profile shape is a function of the distribution in lag times of the individual pellets.

It has been discovered by the inventors herein that the range of pulses from individual pulsed-release pellets is particularly suitable to manipulating active agent release profiles of dosage forms. Blending two ensembles of pulsed-release pellets with different dissolution profiles broadens the distribution of individuals, the sum of which distribution then has a unique slope, or rate of release compared to the individual ensembles. Thus, the resulting combination ensemble has a unique slope compared to its two component ensembles. Blending of two ensembles of pellets does not change the behavior of any individual pellet, but instead provides a combination ensemble with a release profile that cannot be achieved by one population of pellets alone. In one embodiment, the combination ensemble comprises at least two individual ensembles of pulsed-release pellets, wherein each individual ensemble comprises a different coating weight. Combination ensembles of pulsed-release pellets with different coating weights results in ensemble release of a wider distribution of individuals, which in turn alters the rate of release from the combination ensemble to a value not obtainable from a single coat weight itself. In another embodiment, the first and second ensembles comprise different pulsed-release coating compositions, optionally in different weight percents.

A method of optimizing a dissolution profile for a selected active agent dosage form comprises combining a first amount of a first ensemble of pulsed-release pellets having a first dissolution profile with a first T50 and a second amount of a second ensemble of pulsed-release pellets having a second dissolution profile with a second T50 to produce a combination ensemble of pellets having a combination dissolution profile of a combination slope. The slope of the combination dissolution profile corresponds to a single phase release, and the slope of the combination dissolution profile is greater than 10% lower than the slope of the first dissolution profile and greater than 10% lower than the slope of the second dissolution profile. The slope of the combination dissolution profile is one that cannot be achieved by a single coating weight of pulsed-release pellets alone, that is, a broadening of the distribution of pulsed-releases is required to achieve the slope of the combination dissolution profile. The first ensemble of pellets and the second ensemble of pellets comprise a core having disposed thereon a core composition layer, the core composition layer comprising the active pharmaceutical ingredient, and a pulsed-release coating disposed on the core composition layer. The ratio of the first ensemble to the second ensemble to give the desired combination ensemble dissolution can be determined empirically from the dissolution profiles of the first and second ensembles.

The dosage form optionally comprises a third ensemble of pulsed-release pellets comprising a third average pulsed-release coating weight and having a third dissolution profile with a third T50, wherein the third T50 is greater than the first T50 and less than the second T50, that is, the third T50 is intermediate to the first and second T50s. Further, the dosage form optionally comprises a fourth ensemble of pulsed-release pellets comprising a fourth average pulsed-release coating weight and having a fourth dissolution profile with a fourth T50, wherein the fourth T50 is greater than the first T50 and less than the second T50, that is, the fourth T50 is intermediate to the first and second T50s.

In one embodiment, the T50 of the first ensemble of pulsed-release pellets and the T50 of the second ensemble of pulsed-release pellets differ by about 1 to about 4 hours under a specified set of dissolution conditions. In practice, this means that the pulses from the two ensembles are close enough that the individual populations overlap sufficiently to not be able to assign individual pellet release profiles to one population or the other, yet are different enough to give a unique dissolution profile, that is, a unique combination of lag, slope and T50, when combined as a combination ensemble.

In another embodiment, the first ensemble of pulsed-release pellets comprises a first average coating weight, the second ensemble of pulsed-release pellets comprises a second average coating weight, wherein the first average coating weight and the second average coating weight differ by 1 wt % or greater, and wherein coating weights are based on the total weight of the coated pellets in each ensemble. In other embodiments, the coating weights of the two ensembles of pulsed-release pellets differ by 2 wt %, 3 wt %, 4 wt %, 5 wt %, 6 wt %, 7 wt %, 8 wt %, and 9 wt %, based on the weight of the coating material. In this embodiment, the difference in coating weights results in different releases for the ensembles of pellets.

In one embodiment, the method further comprises, prior to combining the two ensembles of pellets, determining the release of at least a portion of the individual pellets in the first ensemble of pulsed-release pellets, determining the release of at least a portion of the individual pellets in the second ensemble of pulsed-release pellets, and determining from the release of individual pellets in the first and second ensembles the first amount of the first ensemble of pulsed-release pellets and the second amount of the second ensemble of pulsed-release pellets to give the optimized in vitro dissolution profile. By determining the breadth of the distribution of release of individual pellets in two ensembles of pellets, one can determine a suitable ratio of the two ensembles to give a blend having a desired slope.

A method of increasing patient compliance comprises providing a dosage form to a human patient in need thereof, wherein the dosage form is produced by the above-described method.

A dosage form comprises a first amount of a first ensemble of pulsed-release pellets comprising a first average pulsed-release coating weight and having a first dissolution profile with a first T50, and a second amount of a second ensemble of pulsed-release pellets comprising a second average pulsed-release coating weight and having a second dissolution profile with a second T50, wherein the first ensemble and the second ensemble are combined to form a combination ensemble of pellets having a combination dissolution profile of a combination slope. The first average pulsed-release coating weight and the second average pulsed-release coating weight differ by 1 wt % or greater, wherein coating weights are based on the total weight of the coated pellets. In this embodiment, the combination slope corresponds to a single phase release, and wherein the combination slope is greater than 10% lower than the slope of the first dissolution profile and greater than 10% lower than the slope of the second dissolution profile, and wherein the first ensemble of pellets and the second ensemble of pellets comprise a core having disposed thereon a core composition layer, the core composition layer comprising the active pharmaceutical ingredient, and the pulsed-release coating disposed on the core composition layer.

The dosage form optionally comprises a third ensemble of pulsed-release pellets comprising a third average pulsed-release coating weight and having a third dissolution profile of a third T50, wherein the third T50 is greater than the first T50 and less than the second T50, that is, the third T50 is intermediate to the first and second T50s. Further, the dosage form optionally comprises a fourth ensemble of pulsed-release pellets comprising a fourth average pulsed-release coating weight and having a fourth dissolution profile of a fourth T50, wherein the fourth T50 is greater than the first T50 and less than the second T50, that is, the fourth T50 is intermediate to the first and second T50s.

In one embodiment, the pulsed-release pellets comprise a core having disposed thereon a core composition layer comprising an active agent and optionally a binder. In one embodiment, the core composition layer is disposed directly on the surface of the core. Exemplary cores include inert spheroids, Nonpareils, sugar spheroids, CELLETS®, CELSPHERE®, microcrystalline cellulose spheres, spheres made of microcrystalline cellulose and one or more sugars, such as lactose, and combinations comprising one or more of the foregoing cores. In one embodiment, the core is a sugar sphere. The size of cores may be, for example, about 250 μm to about 1500 μm. Commercially available sugar spheres are in US standard sieve size ranges of 14-16, 16-18, 18-20, 20-25, 25-30, 30-35, 40-60, for example. The cores comprise about 10 wt % to about 98 wt %, specifically about 20 wt % to about 90 wt %, and more specifically about 30 wt % to about 85 wt %, of the total weight of the core and the core composition layer.

The pellets may be formed by coating (e.g., spraying) the sugar spheres with an aqueous or non-aqueous suspension that comprises the active agent. The active agent may be coated onto the sugar spheres in the presence of, for example, a binder, a filler, a solubilizer, and other additives, and combinations comprising one or more of the foregoing additives. The binder may be, for example, polyethylene oxide, hydroxypropylcellulose, hydroxypropylmethylcellulose, ethylcellulose, cellulose acetate butyrate, hydroxypropylmethylcellulose phthalate, polyvinyl acetate phthalate, acacia, carboxymethylcellulose sodium, dextrin, gelatin, glucose, guar gum, hydroxyethyl, methylcellulose, polymethacrylates, povidone, pregelatinized starch, sodium alginate, zein, and the like, and combinations comprising one or more of the foregoing binders. The binder may comprise, for example, hydroxypropylcellulose, such as hydroxypropylcellulose NF 75-150 cps. The suspension medium may comprise, for example, a solvent such as isopropyl alcohol, ethanol, water, and the like, and combinations comprising one or more of the foregoing solvents. The optional binder, when present, comprises about 0.1 wt % to about 20 wt %, specifically about 0.2 wt % to about 10 wt %, and more specifically about 3 wt % to about 8 wt %, of the total weight of the core and the core composition layer.

In one embodiment, the core is substantially free of an organic acid, i.e., the amount of such organic acid, if any, is sufficiently small so as not to substantially affect the release rate of the active pharmaceutical ingredient from the core. Organic acids include, for example, adipic acid, ascorbic acid, citric acid, fumaric acid, malic acid, succinic acid, and tartaric acid.

The core composition layer comprises an active agent such as diltiazem, verapamil, propranolol, fluoxetine, venalfaxine, methylphenidate, amphetamines, zolpidem, and galantamine suitably in the form of a pharmaceutically acceptable salt. In one embodiment, the active agent is a water-soluble active agent. In some embodiments, the active agent is soluble in the dispersing agent employed in a rotary granulation process. Other suitable active agents include, anti-inflammatory substances, coronary vasodilators, cerebral vasodilators, peripheral vasodilators, anti-infectives, psychotropics, antimanics, stimulants, anti-histamines, gastro-intestinal sedatives, anti-diarrheal preparations, anti-anginal drugs, vasodilators, antiarrythmics, anti-hypertensive drugs, vasoconstrictors drugs useful to treat migraines, anticoagulants and antithrombotic drugs, analgesics, anti-pyretics, hypnotics, sedatives, anti-emetics, anti-nauseants, anticonvulsants, neuromuscular drugs, hyper- and hypoglycaemic agents, thyroid and antithyroid preparations, diuretics, antipasmodics, uterine relaxants, mineral and nutritional additives, antiobesity drugs, anabolic drugs, erythropoietic drugs, antiasthmatics, expectorants, cough suppressants, mucolytics, antiuricemic drugs and other drugs.

Additional suitable active agents include gastrointestinal sedatives such as metoclopramide and propantheline bromide; antacids such as cimetidine; anti-inflammatory drugs such as phenylbutazone, indomethacin, naproxen, ibuprofen, flurbiprofen, diclofenac, dexamethasone, predinisone and prednisolone; coronary vasodilator drugs such as glyceryl trinitrate, isosorbide dinitrate and pentaerythritil tetranitrate; peripheral and cerebral vasodilators such as soloctidilum, vincamine, naftidorofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotilline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; drugs affecting the rhythm of the heart, such as verapamil, nifedipine, diltiazem, procainamide, disopyramide, bretylium toxylate, quinidine sulfate and quinidine gluconate; drugs used in the treatment of hypertension such as propranolol hydrochloride, guanethidine mono-sulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicyclic acid, acetaminophen, codeine phosphate, codeine sulfate, oxydodone, dihydrocodeine tartrate, oxycodeinone, morphine, heroin, nalbuphine, butorphanol tartrate, pentazocine hydrochloride, cyclazacine, pethidine, buprenorphine, scopolamine and mefanamic acid; anti-epileptic drugs such as phenytoin sodium and sodium valproate; neuromuscular drugs such as dantrolene sodium; substances used in the treatment of diabetes such as tolbutamide, disbenase glucagon and insulin; drugs used in the treatment of thyroid gland disfunction such as triodothyronine, thyroxine and propylthiouracil; diuretic drugs such as furosemide, chlorthalidone, hydrochlorthiazide, spironolactone and trimterene; the uterine relaxant drug ritodrine; appetite suppressants such as fenfluramine hydrochloride, phentermine and diethylproprion hydrochloride; antiasthmatic and bronchodilator drugs such as aminophyline, theophyline, salbutamol, orciprenaline sulphate and terbutaline sulphate; expectorant drugs such as fuaiphenesin; cough suppressants such as dextromethorphan and noscapine; mucolytic drugs such as carbocisteine; adecongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; drug useful for treating Crohn's disease, e.g., 5-amino salicyclic acid, and the like.

In one embodiment, the active agent is present in the dosage form as a pharmaceutical salt. “Pharmaceutically acceptable salts” includes derivatives of the active agent, wherein the active agent is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

The active agent comprises about 20 wt % to about 90 wt %, more specifically about 30 wt % to about 85 wt %, of the total weight of the core and the core composition layer.

The active agent-binder coating mixture may be deposited on the core using a rotary granulation process. In this embodiment, the active agent-binder coating mixture is atomized onto a fluidized bed of cores located in the rotor granulator. Because of the difference in size between the cores and the atomized active agent-binder mixture, the active agent sticks to the cores and the binder retains the active agent on the cores. In the fluidized bed, a rotor-disk granulator makes the cores move with fluid-like motion. As the cores move within the fluidized bed, they are sprayed with the active agent-binder mixture until the desired quantity of active agent is deposited upon the cores. Coating of the active agent and binder may optionally be followed by a drying step.

The core comprising the active agent is then coated with a pulsed-release coating composition. In one embodiment, the pulsed-release coating composition comprises a mixture of a relatively large proportion of a lubricant and a relatively small proportion of a wetting agent in admixture with a minor proportion of a first polymer that is permeable to the active agent and water and a major proportion of a polymer that is less permeable to the active agent and water than the first polymer. No organic acids, such as adipic acid, ascorbic acid, citric acid, fumaric acid, malic acid, succinic acid, tartaric acid and fumaric acid, are required to be included into the coating layer. A suitable permeable first polymer is the cationic polymer synthesized from acrylic and methacrylic acid ester with a low content of quaternary ammonium groups, known as EUDRAGIT® RL manufactured by Degussa. EUDRAGIT® RL is a copolymer of acrylic and methacrylic esters comprising quaternary ammonium groups and having a ratio of quaternary ammonium groups to neutral meth(acrylic) esters of 1:20. In this compound, the ammonium groups give rise to the permeability of the polymer. The permeability of EUDRAGIT® RL is reportedly independent of pH. A suitable less permeable second polymer is another such cationic polymer known as EUDRAGIT® RS manufactured by Degussa. EUDRAGIT® RS is less permeable than EUDRAGIT® RL because EUDRAGIT® RS has fewer ammonium groups. EUDRAGIT® RS is a copolymer of acrylic and methacrylic esters comprising quaternary ammonium groups and having a ratio of quaternary ammonium groups to neutral meth(acrylic) esters of 1:40. The permeability of EUDRAGIT® RS is reportedly independent of pH.

The pulsed-release coating layer may alternatively comprise a mixture of polymers, synthetic and/or naturally occurring, that are freely permeable, slightly permeable, water soluble, water insoluble, and polymers whose permeability and/or solubility is affected by pH. In addition to those referred to above, such suitable polymers for inclusion into the coating layer include EUDRAGIT® S, EUDRAGIT® L, EUDRAGIT® E, polyvinyl alcohol, polyvinylpyrrolidone, and combinations comprising one or more of the foregoing polymers. Commercially available polymeric solutions and/or suspensions may also be employed. These solutions/suspensions may optionally contain plasticizing agents to improve the polymer characteristics of the coating. Examples of such solutions and/or suspensions include EUDRAGIT® RS30D, EUDRAGIT RL® 30D, EUDRAGIT® L 30D, EUDRAGIT® E 12.5, EUDRAGIT® RL 12.5 P, EUDRAGIT® RS 12.5, AQUACOAT® made by FMC Corporation, SURELEASE® made by Colorcon Inc, and combinations comprising one or more of the foregoing. AQUACOAT® is an aqueous polymeric dispersion of ethylcellulose and contains sodium lauryl sulfate and cetyl alcohol. SURELEASE® is an aqueous polymeric dispersion of ethylcellulose and contains dibutyl sebacate, oleic acid, ammoniated water and fumed silica.

In addition to the polymers, the pulsed-release coating layer includes a lubricant and optionally a wetting agent. Suitable lubricants include talc, calcium stearate, colloidal silicon dioxide, glycerin, magnesium stearate, mineral oil, polyethylene glycol, and zinc stearate, aluminum stearate, glyceryl monostearate, cetostearyl alcohol, cetyl alcohol, lanolin alcohols, stearyl alcohol, lecithin, mineral oil, and combinations comprising one or more of the foregoing lubricants.

Suitable wetting agents include sodium lauryl sulfate, acacia, benzalkonium chloride, cetomacrogol emulsifying wax, diethanolamine, docusate sodium, sodium stearate, emulsifying wax, hydroxypropyl cellulose, monoethanolamine, poloxamer, polyoxyethylene alkyl ethers, polyoxyethylene castor oil derivatives, polyoxyethylene sorbitan fatty acid esters, polyoxyethylene stearates, propylene glycol alginate, sorbitan esters and triethanolamine, and combinations comprising one or more of the foregoing wetting agents.

The pulsed-release coating layer of the pellet comprises 2 wt % to 7 wt % of the first polymer more permeable to the active agent, 53 wt % to 59 wt % of the second polymer less permeable to the active agent, 0 wt % to 8 wt % of the plasticizer, 0 wt % to 8 wt % of the wetting agent, and 31 wt % to 35 wt % of the lubricant, expressed as percentages of the total weight of the pulsed-release coating layer.

The pulsed-release coating composition optionally comprises additional fillers such as, for example, talc, kaolin, calcium sulfate, and combinations comprising one or more of the foregoing fillers. In one embodiment the optional additional filler comprises talc. The amount of additional filler is about 15 wt % to about 200 wt %, specifically about 30 wt % to about 100 wt % of the weight of the polymers in the pulsed-release coating composition.

The pulsed-release coating optionally comprises a plasticizer. The plasticizer can be water-soluble or water-insoluble. Exemplary water-soluble plasticizers include triethyl citrate, triacetin, polyethylene glycol, propylene glycol, sorbitol, glycerin, and combinations comprising one or more of the foregoing plasticizers. Exemplary water-insoluble plasticizers include dibutyl sebacate, diethyl phthalate, dibutyl phthalate, tributyl citrate, acetyl tributyl citrate, castor oil, mineral oil, glyceryl monostearate, and combinations comprising one or more of the foregoing plasticizers. The plasticizer comprises about 0 wt % to about 30 wt %, specifically about 5 wt % to about 15 wt %, of the total weight of the pulsed-release coating composition.

The pulsed-release coating composition can be applied to the cores using a coating technique used in the pharmaceutical industry, such as fluid bed coating. Once applied and dried, the polymer content of the pulsed-release coating comprises about 5 wt % to about 35 wt % of the total weight of the coated cores, or about 7 wt % to about 25 wt % of the total weight of the coated cores. In this context, coated core means the pellet comprising the API plus any additional coatings.

The pulsed-release coating may be dried before applying an optional second coating. A color imparting agent may be added to the pulsed-release coating composition or a rapidly dissolving seal coat containing color may be coated over the sustained-release coating layer provided that the seal coat is compatible with and does not affect the dissolution of the sustained-release coating layer. Exemplary film-forming agents include polyvinylpyrollidone, hydroxypropyl cellulose, hydroxypropyl methylcellulose, polyethylene oxide, polyethylene glycol, and combinations comprising one or more of the foregoing film-forming agents.

The multiparticulate dosage form of the active agent is optionally encapsulated in hard gelatin to provide a desired quantity of active agent in an oral dosage form. Alternatively, the multi-particulate dosage form may be made into tablets, for example, by first adding about 40 wt % to about 90 wt % of a solid pharmaceutically acceptable tablet excipient which will form a compressible mixture with the coated cores and which may be formed into a tablet without crushing the coated cores, and optionally an effective amount of a tablet disintegrating agent and a lubricant. The solid pharmaceutically acceptable tablet excipient may comprise, for example, carnuba wax, lactose, dextrose, mannitol, microcrystalline cellulose, kaolin, powdered sucrose, vegetable starches and combinations comprising one or more of the foregoing excipients. The tablet disintegrant may comprise crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, hydrogenated vegetable oil (e.g., Lubritab®), zinc stearate, and combinations comprising one or more of the foregoing lubricants.

Also included are methods of increasing patient compliance by administering dosage forms made by the disclosed method.

### EXAMPLE

The following examples further illustrate the invention but, of course, should not be construed as in any way limiting its scope. In particular, the processing conditions are merely exemplary and can be readily varied by one of ordinary skill in the art.

To form pulsed-release pellets, hydroxypropylcellulose (650 g) was dissolved in alcohol (17,000 g). Diltiazem hydrochloride (10,500 g) was suspended in the polymer solution and the entire suspension was applied to sugar spheres (30-35 mesh, 5,000 g) in a rotor granulator. The product was sieved between 500 μm and 1180 μm.

To form the coating for the pulsed-release pellets, a polymer solution was prepared by dissolving EUDRAGIT® RS (83.3 g), EUDRAGIT® RL (5.9 g), triethyl citrate (8.8 g) and sodium dodecylsulfate (2 g) in alcohol (670 g). A portion of the sieved product (700 g) was placed in a Wurster coater and coated with a portion of this solution (655 g) containing suspended talc (43 g). The resulting polymer coated beads were sieved 500 μm to 1180 μm.

Individual pellets were selected at random from the batch for drug release testing. Dissolution of single pellets was performed by circulating 0.1N HCl dissolution media through a sample cell containing a single pellet in line with an HPLC detector. Drug release was monitored by a chart recorder. Response of the chart recorder was calibrated with standard solutions of drug. The volume of the loop was approximately 13 mL. The results are given in FIG. 2.

Samples of 40 mg equivalent and 300 mg equivalent of diltiazem was tested in a Dissolution Apparatus II containing 900 mL of 0.1N HCl media at 37° C. Results are given in FIG. 3. The sum of the individual pellets of FIG. 2 is presented in FIG. 4.

A solution of hydroxypropyl cellulose (HPC) was prepared in ethanol. Diltiazem was suspended in the solution and applied to sugar spheres to a potency of 68%. A solution of PEO in ethanol/water was prepared. Diltiazem was suspended in the solution and applied to 5 kg of these cores until the final potency was 85%.

The overall composition of the cores is presented in Table 1 below.

TABLE 1
Diltiazem Core Formulation

| Item # | Ingredient | Grams | Wt % |
|---|---|---|---|
| 1 | Sugar Sphere (30–35 mesh) | 1076.0 | 8.7 |
| 2 | Polyethylene Oxide (PEO) | 593.0 | 4.7 |
| 3 | Hydroxypropyl Cellulose | 204.0 | 1.6 |
| 4 | Diltiazem HCI | 10620.0 | 85.00 |
| | Total | 12493.0 | 100.00 |
| 1 | Ethanol | 20000.0 | |
| 2 | Water | 3500.0 | |

To two separate lots of 3 kg of these cores was sprayed different quantities of a polymer solution with suspended solids which was prepared as shown in Table 2.

TABLE 2
Coating Composition for the Cores - 3 Kgs

| | | | Grams, | Grams, |
|---|---|---|---|---|
| ITEM # | INGREDIENTS | % | batch 1 | batch 2 |
| | Total Polymer Solids | | | |
| 1 | EUDRAGIT ® RS PO | 75.00 | 170 | 415 |
| 2 | EUDRAGIT ® RL PO | 15.00 | 34 | 83 |
| 3 | Triethyl Citrate | 9.00 | 20 | 50 |
| 4 | Sodium Lauryl Sulfate | 1.00 | 2 | 5 |
| | Total | 100.00 | | |
| | Solids added after dissolving | | | |
| | Polymer (Calculated as 50% of | | | |
| | Polymer Solids) | | | |
| 5 | Colloidal Silicon dioxide | 20.00 | 45 | 110 |
| 6 | Talc | 30.00 | 65 | 165 |
| | Total | 50.00 | | |

The batch 1 (about 7 wt % coating weight) and batch 2 (about 15 wt % coating weight) pellets were blended in a 1:1 ratio. The resulting blend had the dissolution profile shown in FIG. 3, measured in a USP Apparatus-2, pH 6.8 (phosphate buffer), 900 ml, 100 rpm, 37° C.

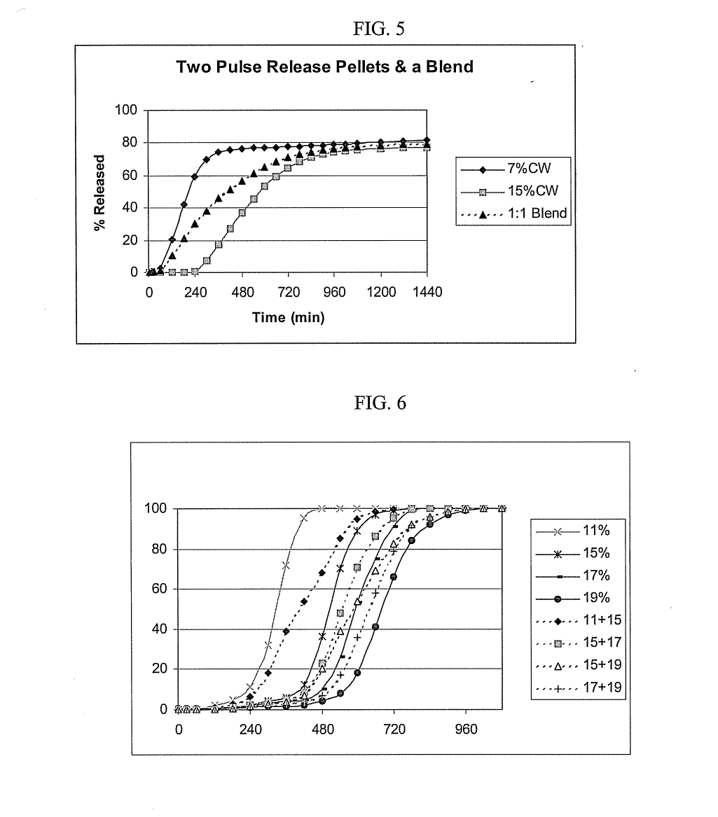

FIG. 5 shows the dissolution profiles for two ensembles of pellets having different coating weights, 7 wt % and 15 wt %. Blending the two pellet ensembles with different dissolution profiles broadens the distribution of individuals, altering the rate of release.

Propranolol pulsed-released pellets were made with cores as given in Table 3 and a coating composition as given in Table 4. Table 5 shows the coating for a 19 wt % coated pellet. Coated pellets were made by rotor granulation to apply drug to sugar spheres, and Wurster coating to apply the coat.

TABLE 3
Propranolol Cores with polyethylene oxide as binder

| Material | Amount |
|---|---|
| Sugar Spheres (20–25 mesh) | 5050 |
| Alcohol SDA3A | 12810 |
| Propranolol HCl | 9300 |
| water | 4270 |
| Polyethylene Oxide | 650 |

TABLE 4
Coating Solution for Pellets

| Material | Amount |
|---|---|
| EUDRAGIT ® RS | 2200 |
| EUDRAGIT ® RL | 155 |
| Triethyl Citrate | 232 |
| Sodium Lauryl Sulfate | 53 |
| Alcohol SDA3A | 17360 |

TABLE 5
Propranolol Pellets

| Material | 19% CW | 17% CW | 15% CW | 11% CW |
|---|---|---|---|---|
| Cores made in Table 3 | 700 | 700 | 700 | 700 |
| Coating Solution made in | 1244 | 1158 | 998 | 699 |
| Table 4 | | | | |
| Talc | 82 | 76 | 66 | 46 |

FIG. 6 shows the release profiles for propranolol pellets having four different coating weights measured at pH 6.8, and theoretical dissolution profiles for four blends each comprising two different coating weights of pellets. The slopes of the individual pellets and blends are given in Table 6:

TABLE 6
Release of propranolol pellets

| Coating weight | Slope of release profile | slope of release profile - 10% |
|---|---|---|
| 11 | 0.667 | 0.6003 |
| 15 | 0.567 | 0.5103 |
| 17 | 0.408 | 0.3672 |
| 19 | 0.4 | 0.36 |
| 11 + 15 | 0.272 | |
| 15 + 17 | 0.353 | |
| 15 + 19 | 0.269 | |
| 17 + 19 | 0.345 | |

Verapamil pulsed-released pellets were made with cores as given in Table 7 and a coating composition as given in Table 4. Coated pellets were made by rotor granulation to apply drug to sugar spheres, and Wurster coating to apply the coat. Table 8 shows the coating for an 11 wt % coated pellet and Table 9 shows the coating for a 19 wt % coated pellet.

TABLE 7
Verapamil Cores

| Material | Amount |
|---|---|
| Alcohol SDA3A | 16920 |
| Hydroxypropyl Cellulose | 650 |
| Sugar Spheres (30–35 mesh) | 4975 |
| Verapamil HCl | 10000 |

TABLE 8
Verapamil Pellets

| Material | 11% CW | 15% CW | 19% CW |
|---|---|---|---|
| Cores made in Table 7 | 700 | 700 | 700 |
| Coating Solution made in Table 4 | 650 | 998 | 1244 |
| Talc | 43 | 66 | 82 |

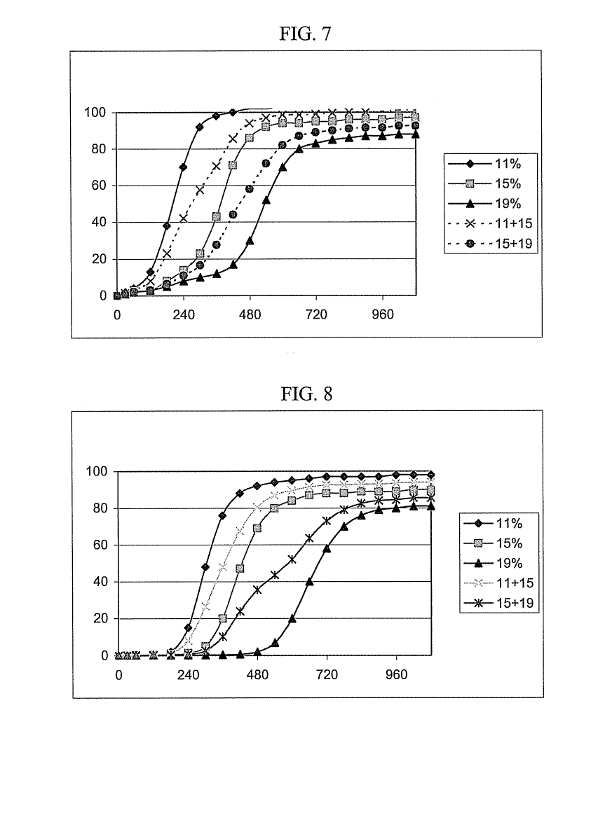

FIG. 7 shows the release profiles at pH 6.8 for verapamil pellets having three different coating weights, and theoretical release profiles for two blends each comprising two different coating weights of pellets. The slopes of the individual pellets and blends are given in Table 9:

TABLE 9

| Coating weight | Slope of release profile | Slope of release profile - 10% |
|---|---|---|
| 11 | 0.533 | 0.4797 |
| 15 | 0.467 | 0.4203 |
| 19 | 0.367 | 0.3303 |
| 11 + 15 | 0.263 | |
| 15 + 19 | 0.246 | |

Diltiazem pulsed-released pellets were made with cores as given in Table 10.

TABLE 10
Diltiazem Cores

| Material | 11% CW |
|---|---|
| Diltiazem HCl | 10050 |
| Sugar Spheres (30–35 mesh) | 5000 |
| Hydroxypropyl Cellulose | 653 |
| Alcohol SDA3A | 17000 |

A coating composition as given in Table 4 was applied to the diltiazem cores as given in Table 11. Coated pellets were made by rotor granulation to apply drug to sugar spheres, and Wurster coating to apply the coat.

TABLE 11
Diltiazem Pellets

| Material | 11% CW | 15% CW | 19% CW |
|---|---|---|---|
| Cores made in Table 10 | 700 | 700 | 700 |
| Coating Solution made in Table 4 | 650 | 998 | 1244 |
| Talc | 43 | 66 | 82 |

FIG. 9 shows the release profiles at pH 6.8 for pellets having three different coating weights, and theoretical profiles for two blends each comprising two different coating weights of pellets. The slopes of the individual pellets and blends are given in Table 12:

TABLE 12

| Coating weight | Slope of release profile | Slope of release profile - 10% |
|---|---|---|
| 11 | 0.508 | 0.4572 |
| 15 | 0.408 | 0.3672 |
| 19 | 0.316 | 0.2844 |
| 11 + 15 | 0.332 | |
| 15 + 19 | 0.169 | |

Disclosed herein are methods of creating dosage forms having unique dissolution profiles by combining ensembles of pellets having dissolution profiles of different slopes. The release of the blended profile is not obtainable from a single coating weight of pellets, for example. The release of the blend is a single phase release due to the overlap in the release of the two pellet ensembles combined to form the blend.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of optimizing a dissolution profile for a selected active agent dosage form, comprising:
combining a first amount of a first ensemble of pulsed-release pellets having a first dissolution profile with a first T50 and a second amount of a second ensemble of pulsed-release pellets having a second dissolution profile with a second T50 to produce a combination ensemble of pellets having a combination dissolution profile of a combination slope,
wherein the combination slope corresponds to a single phase release, and wherein the combination slope is greater than 10% lower than the slope of the first dissolution profile and greater than 10% lower than the slope of the second dissolution profile, and
wherein the first ensemble of pellets and the second ensemble of pellets comprise a core having disposed thereon a core composition layer, the core composition layer comprising the active agent, and a pulsed-release coating disposed on the core composition layer.

2. The method of claim 1, further comprising combining a third ensemble of pulsed-release pellets comprising a third average pulsed-release coating weight and having a third dissolution profile with a third T50, wherein the third T50 is greater than the first T50 and less than the second T50.

3. The method of claim 2, further comprising combining a fourth ensemble of pulsed-release pellets comprising a fourth average pulsed-release coating weight and having a fourth dissolution profile with a fourth T50, wherein the fourth T50 is greater than the first T50 and less than the second T50.

4. The method of claim 1, wherein the T50 of the first ensemble of pulsed-release pellets and the T50 of the second ensemble of pulsed-release pellets differ by about 1 to about 4 hours.

5. The method of claim 1, wherein the first ensemble of pulsed-release pellets comprises a first average coating weight, wherein the second ensemble of pulsed-release pellets comprises a second average coating weight, wherein the first average coating weight and the second average coating weight differ by 1 wt % or greater, based on the weight of coating material.

6. The method of claim 1, wherein the first ensemble of pulsed-release pellets comprises a first average coating weight, wherein the second ensemble of pulsed-release pellets comprises a second average coating weight, wherein the first average coating weight and the second average coating weight differ by 2 wt % or greater, based on the weight of coating material.

7. The method of claim 1, wherein the pulsed-release coating comprises 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active agent, the second copolymer being less permeable to water and the active agent than the first copolymer.

8. The method of claim 1, wherein the active agent comprises diltiazem, verapamil, propranolol, fluoxetine, venalfaxine, methylphenidate, amphetamines, zolpidem, or galantamine.

9. A method of increasing patient compliance, comprising providing an active agent dosage form to a human patient in need thereof, wherein the dosage form is produced by a method comprising:
combining a first amount of a first ensemble of pulsed-release pellets having a first dissolution profile with a first T50 and a second amount of a second ensemble of pulsed-release pellets having a second dissolution profile with a second T50 to produce a combination ensemble of pellets having a combination dissolution profile of a combination slope,
wherein the combination slope corresponds to a single phase release, and wherein the combination slope is greater than 10% lower than the slope of the first dissolution profile and greater than 10% lower than the slope of the second dissolution profile, and
wherein the first ensemble of pellets and the second ensemble of pellets comprise a core having disposed thereon a core composition layer, the core composition layer comprising the active agent, and a pulsed-release coating disposed on the core composition layer.

10. The method of claim 9, further comprising combining a third ensemble of pulsed-release pellets comprising a third average pulsed-release coating weight and having a third dissolution profile with a third T50, wherein the third T50 is greater than the first T50 and less than the second T50.

11. The method of claim 10, further comprising combining a fourth ensemble of pulsed-release pellets comprising a fourth average pulsed-release coating weight and having a fourth dissolution profile with a fourth T50, wherein the fourth T50 is greater than the first T50 and less than the second T50.

12. The method of claim 9, wherein the T50 of the first ensemble of pulsed-release pellets and the T50 of the second ensemble of pulsed-release pellets differ by about 1 to about 4 hours.

13. The method of claim 9, wherein the first ensemble of pulsed-release pellets comprises a first average coating weight, wherein the second ensemble of pulsed-release pellets comprises a second average coating weight, wherein the first average coating weight and the second average coating weight differ by 2 wt % or greater, and wherein coating weights are based on the total weight of the coated pellets in each ensemble.

14. The method of claim 9, wherein the first ensemble of pulsed-release pellets comprises a first average coating weight, wherein the second ensemble of pulsed-release pellets comprises a second average coating weight, wherein the first average coating weight and the second average coating weight differ by 4 wt % or greater, and wherein coating weights are based on the total weight of the coated pellets in each ensemble.

15. The method of claim 9, wherein the pulsed-release coating comprises 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active agent, the second copolymer being less permeable to water and the active agent than the first copolymer.

16. The method of claim 9, wherein the active agent comprises diltiazem, verapamil, propranolol, fluoxetine, venalfaxine, methylphenidate, amphetamines, zolpidem, or galantamine.

17. An active agent dosage form comprising,
a first amount of a first ensemble of pulsed-release pellets comprising a first average pulsed-release coating weight and having a first dissolution profile with a first T50, and
a second amount of a second ensemble of pulsed-release pellets comprising a second average pulsed-release coating weight and having a second dissolution profile with a second T50,
wherein the first ensemble and the second ensemble are combined to form a combination ensemble of pellets having a combination dissolution profile of a combination slope,
wherein the first average pulsed-release coating weight and the second average pulsed-release coating weight differ by 1 wt % or greater based on the weight of the coating material,
wherein the combination slope corresponds to a single phase release, and wherein the combination slope is greater than 10% lower than the slope of the first dissolution profile and greater than 10% lower than the slope of the second dissolution profile, and
wherein the first ensemble of pellets and the second ensemble of pellets comprise a core having disposed thereon a core composition layer, the core composition layer comprising the active agent, and the pulsed-release coating disposed on the core composition layer.

18. The dosage form of claim 17, wherein the T50 of the first ensemble of pulsed-release pellets and the T50 of the second ensemble of pulsed-release pellets differ by about 1 to about 4 hours.

19. The dosage form of claim 17, wherein the pulsed-release coating on the first and second ensemble of pellets comprises 31% to 35% lubricant, 2% to 7% of a first copolymer of acrylic and methacrylic acid esters, and 53% to 59% of a second copolymer of acrylic and methacrylic acid esters, expressed as percentages of the total weight of the pulsed-release coating layer, the first copolymer being permeable to water and the active pharmaceutical ingredient, the second copolymer being less permeable to water and the active pharmaceutical ingredient than the first copolymer.

20. The dosage form of claim 17, wherein the active agent comprises diltiazem, verapamil, propranolol, fluoxetine, venalfaxine, methylphenidate, amphetamines, zolpidem, or galantamine.
